(19) United States
(12) Patent Application Publication
(10) Pub. No.: US 2021/0275105 A1
(43) Pub. Date: Sep. 9, 2021

(54) VITAL SIGN SENSING METHOD AND SYSTEM USING A COMMUNICATION DEVICE

(71) Applicant: National Kaohsiung University of Science and Technology, Kaohsiung City (TW)

(72) Inventors: Kang-Chun Peng, Kaohsiung City (TW); Jen-Hao Lee, Kaohsiung City (TW)

(21) Appl. No.: 16/850,162

(22) Filed: Apr. 16, 2020

(30) Foreign Application Priority Data
Mar. 4, 2020 (TW) 109107018

(51) Int. Cl. A61B 5/00; A61B 5/0205

(52) U.S. Cl. CPC A61B 5/7228; A61B 5/7221; A61B 5/0022; A61B 5/0205; A61B 5/7235

(57) ABSTRACT

Vital sign sensing method and system using a communication device are disclosed in the present invention. An EVM algorithm is performed on a demodulated in-phase signal and a demodulated quadrature-phase signal output from an IQ demodulator for extracting a vital-sign signal of a subject. Any communication device can still preserve communication function while being used as a vital sign sensor, no other hardware architecture is required. The vital sign sensing method and system can overcome the shortcoming of signal interference between the conventional active sensing system and the communication device and also can reduce the construction costs of noncontact vital sign sensing system significantly.

### FIELD OF THE INVENTION

This invention generally relates to vital sign sensing method and system, and more particularly to vital sign sensing method and system using a communication device.

### BACKGROUND OF′ THE INVENTION

Conventional noncontact vital-sign sensor exploits the Doppler Effect between wireless signals emitted from radar and human body to detect vital signs (e.g. respiration and heartbeat). However, it is difficult to identify the Doppler phase shifts in the wireless signals because the vibration caused by vital sign of the human body is tiny. In order to monitor tiny vibration of vital sign, conventional radar equipped with additional active circuit for pure sine wave generation is required, but construction costs and power consumption may be increased.

U.S. Pat. No. 9,846,226, entitled “Motion detection device”, discloses a motion detection device capable of detecting gesture by using the surrounding communication signals. With reference to FIG. 3 of U.S. Pat. No. 9,846,226, the wireless signal received by the antenna 110 and coupled by the coupler 150 is injected into the voltage-controlled oscillator 130 to allow the voltage-controlled oscillator 130 to operate in the injection-locked state and has a frequency variation, such that the sensitivity of the radar for sensing the Doppler phase shift of the communication signal caused by the gesture can be increased. U.S. Pat. No. 9,846,226 also discloses the motion detection device can serve as a sensor for short-distance vital sign detection in column 13 lines 45-62, nevertheless, it cannot be used as a vital sign sensor for long-distance detection or under the situation that a barrier exists between receiver and human body.

### SUMMARY

By performing EVM (Error vector magnitude) algorithm on demodulated 1-phase and Q-phase signals, vital sign sensing method and system disclosed in the present invention allows any communication device to be used as highly sensitive sensor of tiny vibration such that vital sign detection is available.

A vital sign sensing method of the present invention includes steps of: transmitting a transmitted signal to a subject by using a transmitter; receiving a reflected signal reflected from the subject as a received signal by using a receiver, an IQ demodulator of the receiver is configured to demodulate the received signal to obtain a demodulated in-phase signal and a demodulated quadrature-phase signal; and receiving the demodulated in-phase signal and the demodulated quadrature-phase signal by using a compute unit, the compute unit is configured to perform an error vector magnitude algorithm on the demodulated in-phase signal and the demodulated quadrature-phase signal to extract a vital-sign signal of the subject.

A vital sign sensing system of the present invention includes a transmitter, a receiver and a compute unit. The transmitter is configured to transmit a transmitted signal to a subject. The receiver includes a receive antenna and an IQ demodulator, the receive antenna is configured to receive a reflected signal reflected from the subject as a received signal, the IQ demodulator is coupled to the receive antenna for receiving the received signal and configured to demodulate the received signal to obtain a demodulated in-phase signal and a demodulated quadrature-phase signal. The compute unit is coupled to the receiver for receiving the demodulated in-phase signal and the demodulated quadrature-phase signal, and configured to perform an EVM algorithm on the demodulated in-phase signal and the demodulated quadrature-phase signal to extract a vital-sign signal of the subject.

In order to extract the vital sign of the subject, the EVM algorithm is performed on the in-phase and quadrature-phase signals demodulated by the IQ demodulator. The present invention can utilize current wireless communication signals to detect vital signs directly, and any current communication device still preserves communication function while being used as vital sign sensor. Signal interference between the vital sign sensing system of the present invention and the communication device is avoided. Furthermore, the present invention overcomes the disadvantages of high costs and high power consumption.

### DESCRIPTION OF THE DRAWINGS

FIG. 1 is a functional block diagram illustrating a vital sign sensing system in accordance with a first embodiment of the present invention.

FIG. 2 is a functional block diagram illustrating a vital sign sensing system in accordance with a second embodiment of the present invention.

FIG. 3 is a functional block diagram illustrating a vital sign sensing system in accordance with a third embodiment of the present invention.

FIG. 4 is a functional block diagram illustrating a vital sign sensing system in accordance with a fourth embodiment of the present invention.

FIG. 5 is a layout illustrating a first scenario of the present invention.

FIG. 6 displays EVM and vital signs detected by the first scenario of the present invention using 64-QAM modulation.

FIG. 7 displays EVM and vital signs detected by the first scenario of the present invention using QPSK modulation.

FIG. 8 is a layout illustrating a second scenario of the present invention.

FIG. 9 displays EVM and vital signs detected by the second scenario of the present invention using 64-QAM modulation.

FIG. 10 displays EVM and vital signs detected by the second scenario of the present invention using QPSK modulation.

### DETAILED DESCRIPTION OF THE INVENTION

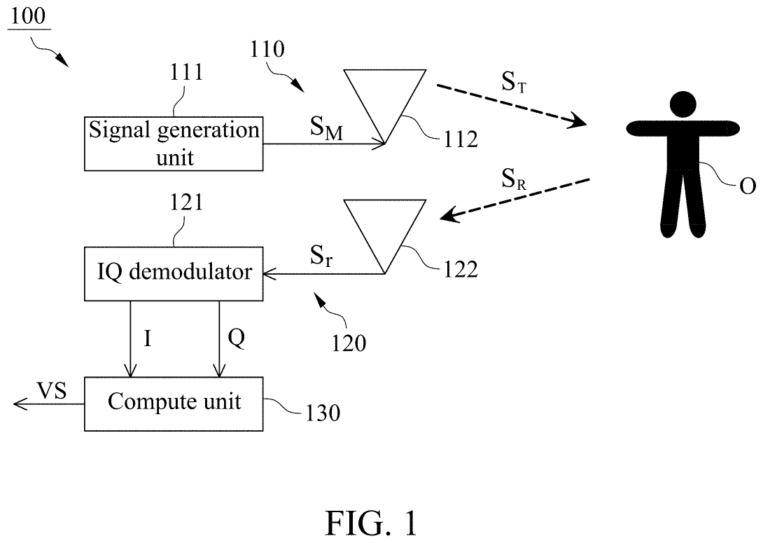

With reference to FIG. 1, a vital sign sensing system 100 in accordance with a first embodiment of the present invention includes a transmitter 110, a receiver 120 and a compute unit 130. In the first embodiment, the transmitter 110 and the receiver 120 are mounted in the same communication device. The communication device can be any commercial wireless communication device, such as mobile phone, laptop, Wi-Fi access point and mobile communication base station, that is connected to the network according to communication protocol standards.

The transmitter 110 of the first embodiment includes a signal generation unit 111 and a transmit antenna 112. The signal generation unit 111 having signal generator, mixer and modulator is configured to generate and transmit a modulated signal S_{M} to the transmit antenna 112, and the transmit antenna 112 is configured to transmit the modulated signal S_{M} to the environment as a transmitted signal S_{T}. The transmitted signal S_{T} is represented as follows:

S_{Tx}(t)=[I_{Tx}(t)+jQ_{Tx}(t)]e^{jω}^{_{c}}^{t}

where S_{Tx} (t) is the transmitted signal S_{T}, I_{Tx} (t) and Q_{TX} (t) denote the transmitted in-phase and quadrature-phase baseband signals, respectively; and ω_{c} is the carrier frequency of communication signal. The transmitted signal S_{T} is transmitted toward a subject O in the environment, and a reflected signal S_{R} is reflected from the subject O. When the subject O has a motion relative to the transmit antenna 112, the motion of the subject O generates the Doppler effect on the transmitted signal S_{T} such that the reflected signal SR contains the Doppler phase shifts. If the motion of the subject O is caused by vital signs, such as respiration and heartbeat, the reflected signal S_{R} contains the Doppler phase shifts caused by the vital signs.

In the first embodiment, the transmitted signal S_{T} from the transmitter 110 is a wireless communication signal and can be a phase-shift keying signal (e.g. BPSK, QPSK, OQPSK, DQSK, 4/π PSK, 8-PSK, 16-PSK, 32-PSK or is 64-PSK) or a quadrature amplitude modulation signal (e.g. 4-QAM, 8-QAM, 16-QAM, 32-QAM, 128-QAM, 256-QAM or 1024-QAM) available for EVM calculation.

The receiver 120 includes an IQ demodulator 121 and a receive antenna 122 which is configured to receive the reflected signal S_{R} from the subject O as a received signal Sᵣ, the received signal Sᵣ is represented as follows:

$$\begin{matrix}{S}_{rx} ( t ) = {GS}_{Tx} ( t - {τ}_{1} ) {e}^{j {ω}_{c} ( t - {τ}_{1} )} {e}^{j {θ}_{d} ( t - {τ}_{2} )} {e}^{- j {ω}_{c} ( t - {τ}_{1} )} \\ = G [ {I}_{Tx} ( t - {τ}_{1} ) + {jQ}_{Tx} ( t - {τ}_{1} ) ] {e}^{j {θ}_{d} ( t - {τ}_{2} )}\end{matrix}$$

where Sᵣₓ(t) is the received signal Sᵣ, G is the magnitude variation between the transmitted signal S_{T} and the received signal Sᵣ, τ₁ is the propagation time between the transmitter 110 and the receiver 120, τ₂ is the propagation time between the subject O and the receiver 120, and θ_{d} is the Doppler phase shifts caused by the motion of the subject O.

The IQ demodulator 121 is electrically connected to the receive antenna 122 for receiving the received signal Sᵣ and configured to demodulate the received signal Sᵣ to obtain a demodulated in-phase signal I and a demodulated quadrature-phase signal Q. The IQ demodulator 121 includes power splitter, quadrature power splitter and mixer. The circuits in the IQ demodulator 121 is conventional and will not be described here. The demodulated in-phase signal I and the demodulated quadrature-phase signal Q are represented as follows:

Iᵣₓ(t)=GI_{Tx}(t−τ₁)cos[θ_{d}(t−τ₂)]−GQ_{Tx}(t−τ₁)sin[θ_{d}(t−τ₂)]

Qᵣₓ(t)=GQ_{Tx}(t−τ₁)cos[θ_{d}(t−τ₂)]+GI_{Tx}(t−τ₁)sin[θ_{d}(t−τ₂)]

where Iᵣₓ(t) and Qᵣₓ(t) are the demodulated in-phase signal I and the demodulated quadrature-phase signal Q, respectively. And the Doppler phase shifts caused by the motion of the subject O is represented as follows:

$${θ}_{d} ( t ) = \frac{4 π {x}_{d} ( t )}{λ} = \frac{4 π [ {x}_{h} sin ( {ω}_{h} t ) + {x}_{r} sin ( {ω}_{r} t ) ]}{λ}$$

where x_{d} (t) denotes the motion of the subject O, λ denotes the wavelength of the transmitted signal S_{T} in air, xₕ and xᵣ denote the motion of the subject O due to the heartbeat and respiration, respectively, ωₕ and ωᵣ denote the frequency of the heartbeat and respiration, respectively. When the motion of the subject O caused by the heartbeat and the respiration is much smaller than the wavelength of the transmitted signal S_{T}

cos[θ_{d}(t−τ₂)]≃1

sin[θ_{d}(t−τ₂)]θ_{d}(t−τ₂)

the demodulated in-phase signal I and the demodulated quadrature-phase signal Q can be simplified as follows:

Iᵣₓ(t)≃GI_{Tx}(t−τ₁)−GQ_{Tx}(t−τ₁)θ_{d}(t−τ₂)

Qᵣₓ(t)≃GQ_{Tx}(t−τ₁)+GI_{Tx}(t−τ₁)θ_{d}(t−τ₂)

the first terms GI_{Tx}(t−τ₁) and GQ_{Tx}(t−τ₁) in the above equations describe the communication signals, and the second terms GQ_{Tx}(t−τ₁)θ_{d}(t−τ₂) and GIT_{Tx}(t−τ₁)θ_{d}(t−τ₂) in the above equations describe the system noise that is caused by the vital sign of the subject O.

With reference to FIG. 1, the compute unit 130 is electrically connected to the receiver 120 for receiving the demodulated in-phase signal I and the demodulated quadrature-phase signal Q and is configured to perform an EVM algorithm on the demodulated in-phase signal I and the demodulated quadrature-phase signal Q to obtain a vital-sign signal VS of the subject O. In the first embodiment, one step of the EVM algorithm is mapping the demodulated in-phase signal I and the demodulated quadrature-phase signal Q to a constellation diagram by using the compute unit 130 to obtain an ideal vector and an error vector. According to the current symbol rate of the system, the compute unit 130 samples the demodulated in-phase signal I and the demodulated quadrature-phase signal Q to establish an instantaneous constellation diagram. The sampled instantaneous in-phase ideal vector and the sampled instantaneous quadrature-phase ideal vector are represented as follows:

I_{rx,i}(t)=GI_{Tx}(t−τ₁)

Q_{rx,i}(t)=GQ_{Tx}(t−τ₁)

where I_{rx,i}(t) and Q_{rx,i}(t) are the instantaneous in-phase ideal vector and the instantaneous quadrature-phase ideal vector, respectively. And the instantaneous in-phase error vector and the instantaneous quadrature-phase error vector are represented as follows:

ΔIᵣₓ(t)=−GQ_{Tx}(t−τ₁)θ_{d}(t−τ₂)

ΔQᵣₓ(t)=GI_{Tx}(t−τ₁)θ_{d}(t−τ₂)

where ΔIᵣₓ(t) is the instantaneous in-phase error vector and ΔQᵣₓ (t) is the instantaneous quadrature-phase error vector. The ideal vector is synthesized from the instantaneous in-phase ideal vector and the instantaneous quadrature-phase ideal vector, and the error vector is synthesized from the instantaneous in-phase error vector and the instantaneous quadrature-phase error phase. The magnitudes of the ideal vector and the error vector are, respectively,

A_{rx,i}(t)=\sqrt{I_{rx,i}²(t)+Qr_{x,i}²(t)}=G\sqrt{I_{Tx}²(t−τ₁)+Q_{Tx}²(t−τ₁)}

ΔAᵣₓ(t)=\sqrt{ΔIᵣₓ²(t)+ΔQᵣₓ²(t)}=Gθd(t−τ₂)\sqrt{I_{Tx}²(t−τ¹)+Q_{Tx}²(t−τ₁)}

where A_{rx,i}(t) and ΔAᵣₓ (t) denote the magnitudes of the ideal vector and the error vector, respectively. Another step of the EVM algorithm is to calculate a phase variation signal according to the ideal vector and the error vector by using the compute unit 130. The calculation is represented as follows:

$${θ}_{d} ( t - {τ}_{2} ) = \frac{Δ {A}_{r x} ( t )}{{A}_{rx , i} ( t )}$$

where θ_{d}(t−τ₂) is the phase variation signal. The phase variation signal caused by the motion of the subject O with respect to the transmitter 110 is found by dividing the magnitude of the error vector by the magnitude of the ideal vector. Consequently, the compute unit 130 can perform a spectrum analysis on the phase variation signal, such as fast Fourier transform, to extract the vital-sign signal VS of the subject O.

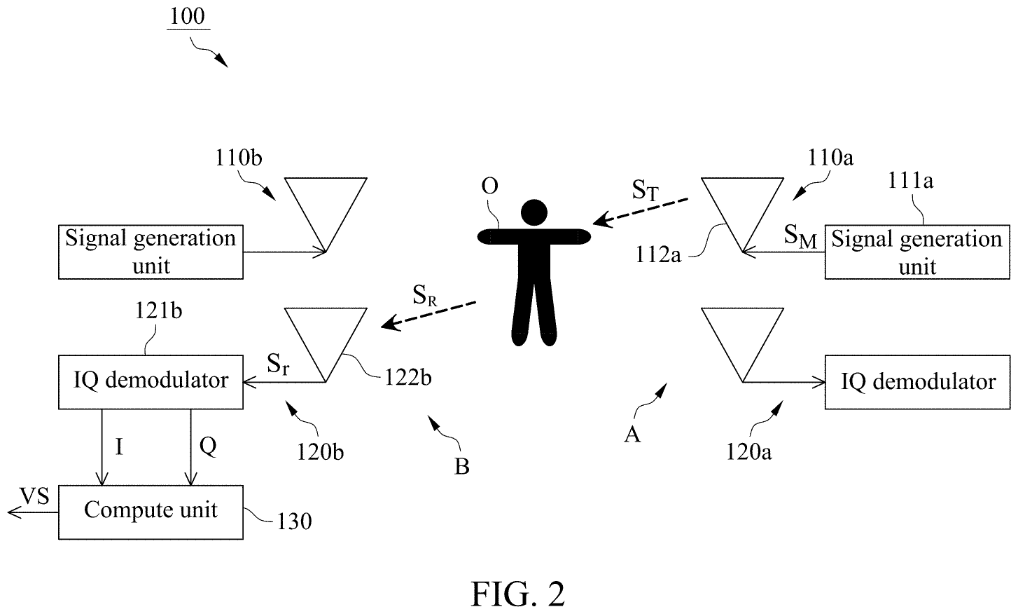

FIG. 2 shows a second embodiment of the present invention. Different to the first embodiment, the transmitter 110a and the receiver 120b of the second embodiment are mounted in a first communication device A and a second communication device B, respectively. The transmitted signal S_{T} is transmitted from the transmitter 110a of the first communication device A to the subject O but the reflected signal S_{R} from the subject O is received by the receiver 1201) of the second communication device B as the received signal Sᵣ. Likewise, the motion of the subject O relative to the transmitter 110a causes the Doppler effect on the transmitted signal S_{T} and causes the reflected signal S_{R} and the received signal Sᵣ both contain the Doppler phase shifts result from the motion of the subject O. The IQ demodulator 121b receives and demodulates the received signal Sᵣ to obtain the demodulated in-phase signal I and the demodulated quadrature-phase signal Q, and the compute unit 130 receives the demodulated in-phase signal I and the demodulated quadrature-phase signal Q and preforms the EVM algorithm on the demodulated signals to extract the vital-sign signal VS of the subject O.

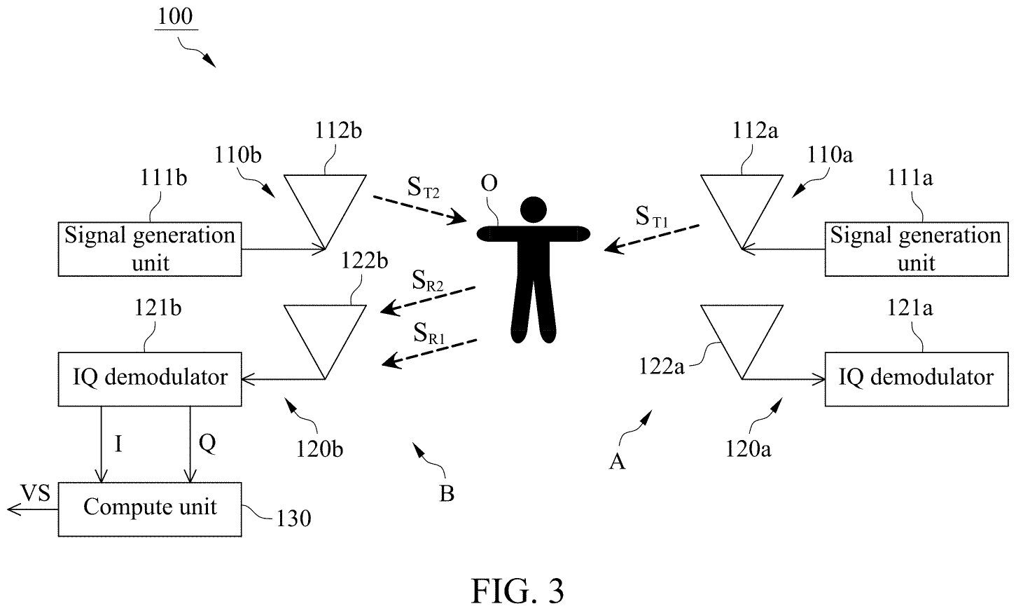

With reference to FIG. 3, it displays a third embodiment of the present invention. Not only the first communication device A transmits a first transmitted signal S_{T1} to the subject O, but also the second communication device B transmits a second transmitted signal S_{T2} to the subject O. The receiver 120b of the second communication device B receives two reflected signals, a first reflected signal S_{R1} and a second reflected signal S_{R2} reflected from the subject O. Random body movements of the subject O cause out-of-phase Doppler phase shifts in the first transmitted signal S_{T1} and the second transmitted signal S_{T2}, on the contrary, vital sign movements of the subject O generate in-phase Doppler phase shifts in the first transmitted signal S_{T1} and the second transmitted signal S_{T2}. The Doppler phase shifts due to body movements in the signals will be cancelled out. Accordingly, the vital sign sensing will not be interfered by the body movements.

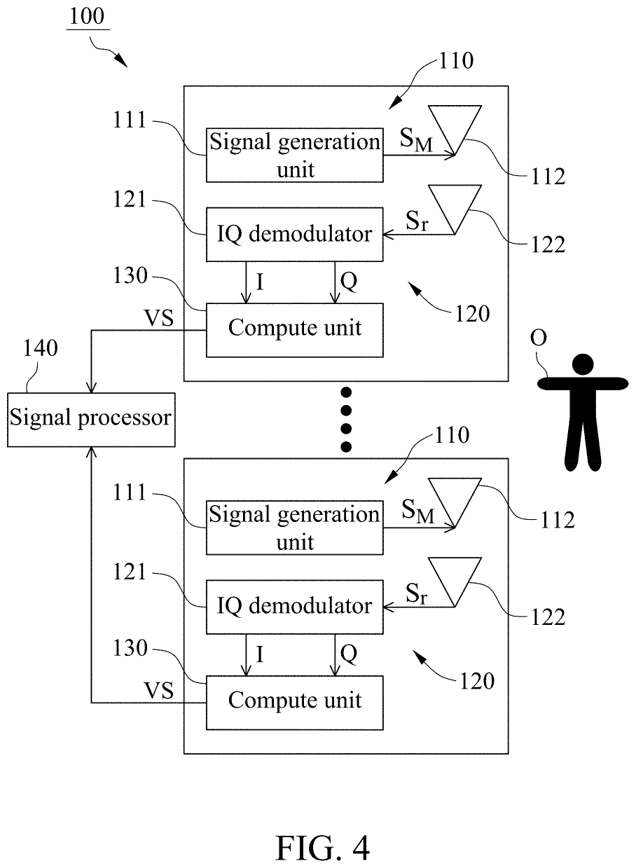

The vital sign sensing 100 as shown in FIG. 4 is a fourth embodiment of the present invention, it includes a plurality of transmitters 110, a plurality of receivers 120, a plurality of compute units 130 and a signal processor 140. Each of the compute units 130 is electrically connected to one of the receivers 120 for receiving the demodulated in-phase signal I and the demodulated quadrature-phase signal Q, and each of the compute units 130 is configured to perform the EVM algorithm on the demodulated in-phase signal I and the demodulated quadrature-phase signal Q to obtain vital-sign signals VS of the subject O. The signal processor 140 is electrically connected to the compute units 130 for receiving the vital-sign signals VS and configured to determine orientation of the subject O according to the vital-sign signals VS. In the fourth embodiment, the transmitted signals S_{T} from the transmitters 110 are delayed to become a beam transmitted toward a specific angle. The beam can be directed toward different positions by adjusting the delay to change the beam angle, and the vital signs VS are extracted by the compute units 130 only when the beam directs toward the subject O so as to determine the orientation of the subject O.

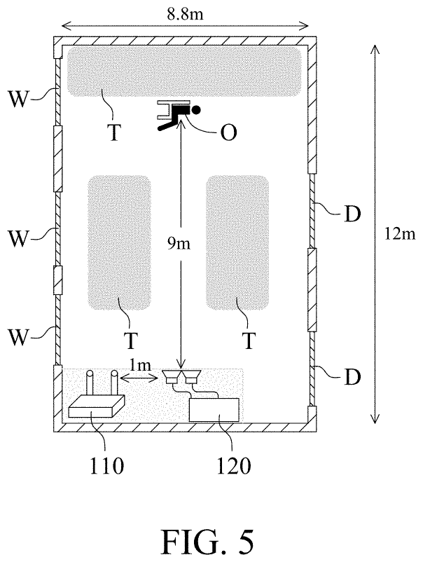

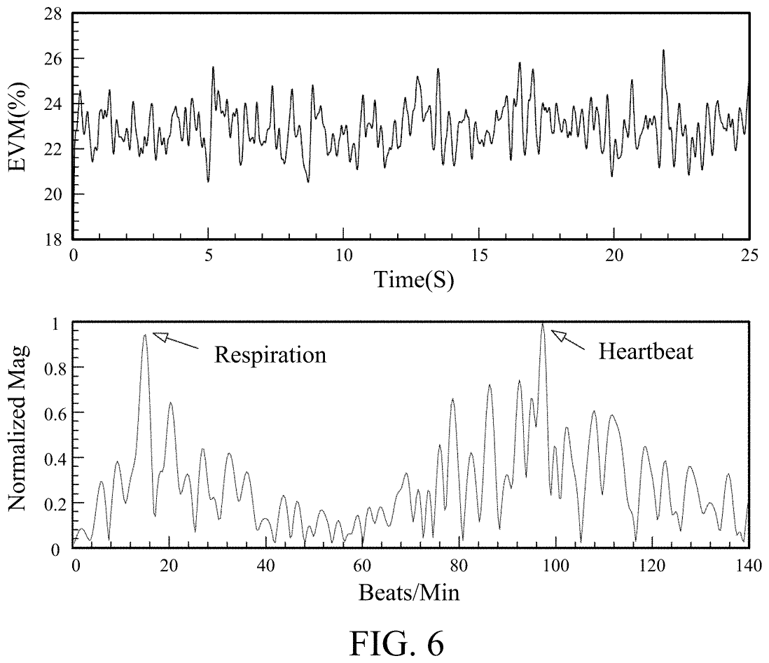

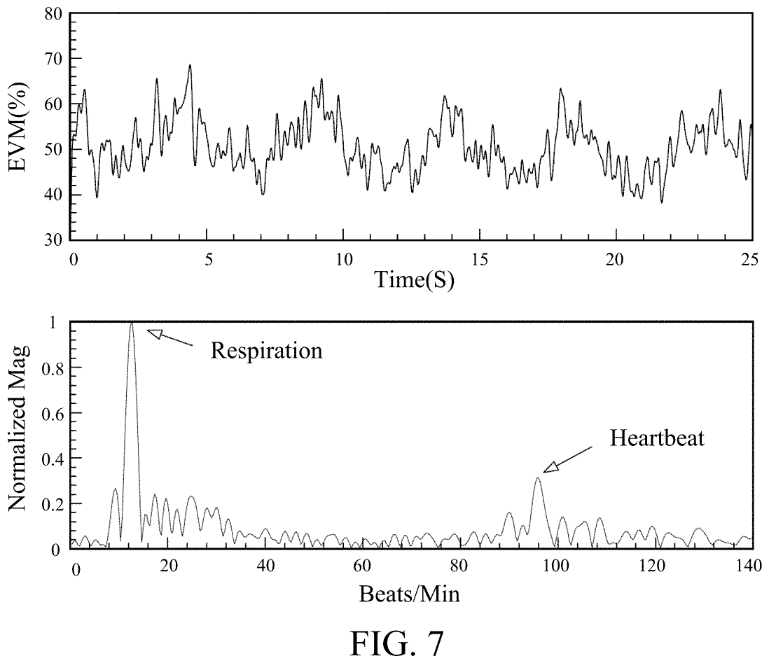

FIG. 5 is first scenario layout of the present invention, where W denotes window and D demotes stainless steel door. In the first scenario, the transmitter 110 is a Wi-Fi access point, the receiver 120 is a universal software radio peripheral (USRP) having two antennas. The transmitter 110 is placed 1 m next to the receive antenna of the receiver 120, and the distance between the seated subject O and the receive antenna is 9 m. FIG. 6 shows the EVM and vital-sign signal detected by the first scenario, in which the modulation type and output power of the transmitted signal from the transmitter 110 are set to 64-QAM and −30 dBm, respectively. Besides, FIG. 7 shows the EVM and vital-sign signal detected by the first scenario that transmits a QPSK-modulated signal with an output power of −30 dBm from the transmitter 110. The vital signs of the respiration and heartbeat can be extracted from the vital-sign signals over long distances by different modulation types.

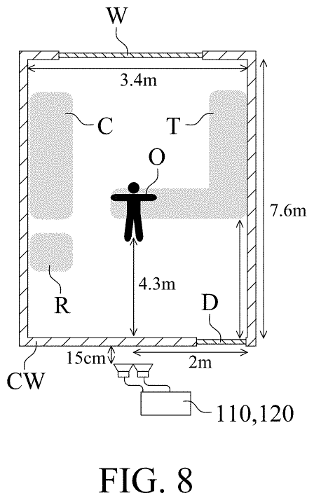

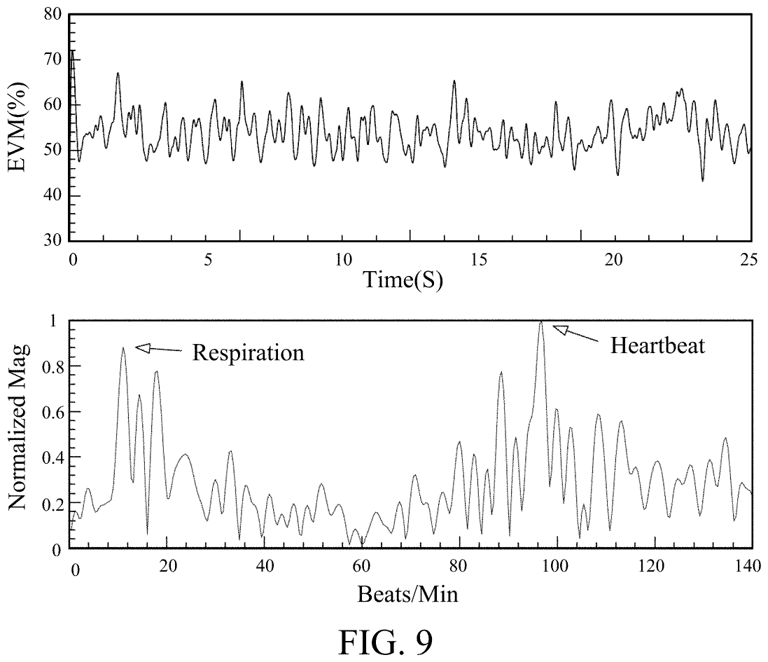

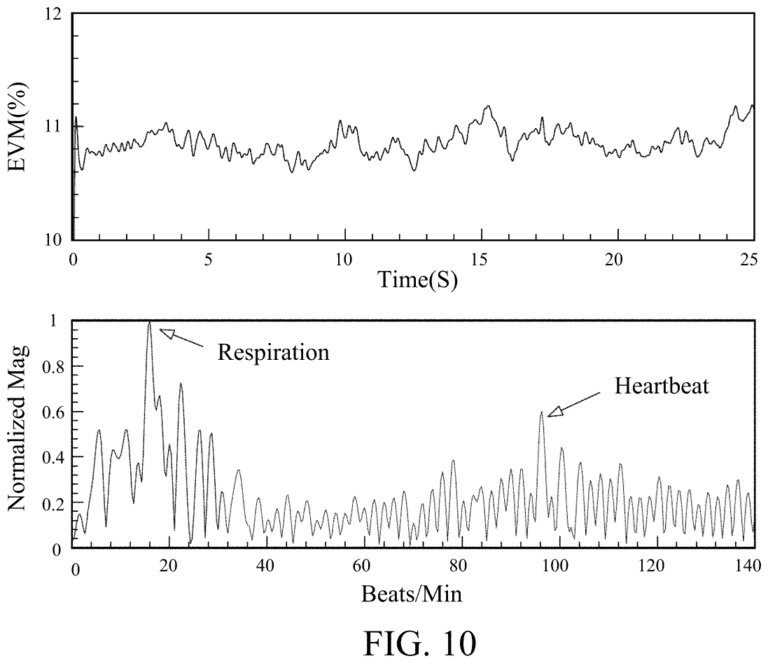

Second scenario layout of the present invention is shown in FIG. 8. W, C, T, R, D and CW denote window stainless steel bookcase, stainless steel table, refrigerator, closed solid wooden door and 15-cm-thick reinforced concrete wall, respectively. The transmitter 110 and the receiver 120 are installed in the USRP having two antennas for transmitting and receiving wireless signals. The reinforced concrete wall CW is located between the transceiver antenna and the standing subject O, the distance from the standing subject O to the reinforced concrete wall CW is 4.3 m, and the two antennas are placed 15 cm out away the reinforced concrete wall CW. FIGS. 9 and 10 show the EVM and is vital-sign signal detected by the second scenario using 64-QAM and QPSK modulations, respectively, with an output power of 10 dBm. The vital signs of the respiration and heartbeat can be extracted from the vital-sign signals using different modulation types with the barrier of the concrete wall.

In the present invention, the EVM algorithm is performed on the in-phase signal I and the quadrature-phase signal Q demodulated by the IQ demodulator 120 to extract the vital-sign signal VS of the subject O. The current wireless communication signals are available to detect the vital-sign signal VS and any communication device can preserve communication function without any signal interference while being used as a vital-sign sensor. Consequently, the vital sign sensing system 100 of the present invention has the advantages of low cost and lower power consumption.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A vital sign sensing method comprising steps of:
transmitting a transmitted signal to a subject by using a transmitter;
receiving a reflected signal reflected from the subject as a received signal by using a receiver, an IQ demodulator of the receiver is configured to demodulate the received signal to obtain a demodulated in-phase signal and a demodulated quadrature-phase signal; and
receiving the demodulated in-phase signal and the demodulated quadrature-phase signal by using a compute unit, the compute unit is configured to perform an error vector magnitude (EVM) algorithm on the demodulated in-phase signal and the demodulated quadrature-phase signal to extract a vital-sign signal of the subject.

2. The vital sign sensing method in accordance with claim 1, wherein the EVM algorithm comprises steps of:
mapping the demodulated in-phase signal and the demodulated quadrature-phase signal to a constellation diagram by using the compute unit to obtain an ideal vector and an error vector; and
calculating a phase variation signal according to the ideal vector and the error vector by using the compute unit.

3. The vital sign sensing method in accordance with claim 2, wherein the compute unit is configured to perform a spectrum analysis on the phase variation signal to extract the vital-sign signal.

4. The vital sign sensing method in accordance with claim 2, wherein the compute unit utilizes equations as follows to map the demodulated in-phase signal and the demodulated quadrature-phase signal to the constellation diagram
A_{Rx,i}(t)=\sqrt{I_{Rx,i}²(t)+Q_{Rx,i}²(t)}
ΔA_{Rx,i}(t)=\sqrt{ΔI_{Rx,i}²(t)+ΔQ_{Rx,i}²(t)}
where A_{Rx,i}(t) is the ideal vector, ΔA_{Rx,i} (t) is the error vector, I_{Rx,i}(t) is an instantaneous in-phase ideal vector, Q_{Rx,i}(t) is an instantaneous quadrature-phase ideal vector, ΔI_{Rx,i}(t) is an instantaneous in-phase error vector, ΔQ_{Rx,i}(t) is an instantaneous quadrature-phase error vector, the instantaneous in-phase ideal vector and the instantaneous in-phase error vector are sampled from the demodulated in-phase signal, and the instantaneous quadrature-phase ideal vector and the instantaneous iQ quadrature-phase error vector are sampled from the quadrature-phase signal.

5. The vital sign sensing method in accordance with claim 4, wherein a calculation of the phase variation signal is represented as follows:
$${θ}_{d} ( t - {τ}_{2} ) = \frac{Δ {A}_{Rx , i} ( t )}{{A}_{Rx , i} ( t )}$$
where θ_{d}(t−τ₂) is the phase variation signal, and τ₂ is a propagation time between the receiver and the subject.

6. The vital sign sensing method in accordance with claim 1, wherein the transmitted signal from the transmitter is a wireless communication signal.

7. A vital sign sensing system comprising:
a transmitter configured to transmit a transmitted signal to a subject;
a receiver including a receive antenna and a IQ demodulator, the receive antenna is configured to receive a reflected signal reflected from the subject as a received signal, the IQ demodulator is coupled to the receive antenna for receiving the received signal and configured to demodulate the received signal to obtain a demodulated in-phase signal and a demodulated quadrature-phase signal; and
a compute unit coupled to the receiver for receiving the demodulated in-phase signal and the demodulated quadrature-phase signal, the compute unit is configured to perform an EVM algorithm on the demodulated in-phase signal and the demodulated quadrature-phase signal to extract a vital-sign signal of the subject.

8. The vital sign sensing system in accordance with claim 7, wherein the transmitter and the receiver are installed in a same communication device or installed in different communication devices.

9. The vital sign sensing system in accordance with claim 7 comprising a plurality of transmitters, a plurality of receivers, a plurality of compute units and a signal processor, wherein the compute units are electrically connected to the receivers, respectively, for receiving demodulated in-phase signals and demodulated quadrature-phase signals from the receivers and configured to perform the EVM algorithm on the demodulated in-phase signals and the demodulated quadrature-phase signals to obtain vital-sign signals of the subject, the signal processor is electrically connected to the compute units for receiving the vital-sign signals and configured to determine a orientation of the subject according to the vital-sign signals.

10. The vital sign sensing system in accordance with claim 7, wherein the transmitted signal from the transmitter is a wireless communication signal.
